(19) United States
(12) Patent Application Publication
(10) Pub. No.: US 2026/0097155 A1
(43) Pub. Date: Apr. 9, 2026

(54) VASCULAR REPAIR DEVICE

(71) Applicant: The Nemours Foundation, Jacksonville, FL (US)

(72) Inventors: David BLAUVELT, Chadds Ford, PA (US); Prasad NITHIANANDAM, Wilmington, DE (US)

(73) Assignee: The Nemours Foundation, Jacksonville, FL (US)

(21) Appl. No.: 19/346,749

(22) Filed: Oct. 1, 2025

Related U.S. Application Data
(60) Provisional application No. 63/704,709, filed on Oct. 8, 2024.

(51) Int. Cl. A61L 31/14; A61B 17/00; A61L 31/04; A61M 1/16; A61M 1/36

(52) U.S. Cl. CPC A61L 31/145; A61B 17/0057; A61L 31/042; A61L 31/048; A61L 31/148; A61M 1/1698; A61M 1/3659

(57) ABSTRACT

A vascular repair device includes a hydrogel substrate configured to be positioned at least partially around a target vessel, a biodegradable adhesive configured to be positioned at least partially around the hydrogel substrate, and a biodegradable mold defining a trough sized to accommodate at least a portion of the target vessel and configured to contain at least a portion of the biodegradable adhesive.

### RELATED APPLICATION

This application is related to and claims priority from U.S. Provisional Application No. 63/704,709 filed Oct. 8, 2024, the disclosure of which is incorporated herein by reference in its entirety,

### FIELD

The present disclosure relates generally to apparatuses, systems, and methods for repairing vessels. More specifically, the disclosure relates to apparatuses, systems, and methods for sutureless repair of vessels including arteries.

### BACKGROUND

Vasculature of a patient may be accessed by a surgeon or medical care provider for various reasons. Vascular access can be implemented for many life-saving procedures, including but not limited to, extracorporeal membrane oxygenation (ECMO), intravascular device delivery and deployment, and so forth. ECMO is an advanced form of life support for patients with pulmonary and/or cardiac failure. However, it requires placement of large cannulas into major vascular structures, often the jugular vein and carotid artery. This can be especially difficult for young patients such as neo-natal patients who generally have small vasculature.

Following patient improvement and discontinuation of ECMO support, the cannulas are removed, leaving a large hole in the accessed blood vessels. While the veins can often heal, the carotid artery requires either microvascular repair or ligation. Repair of the vessel is technically challenging and is associated with short term complications such as thrombosis and subsequent strokes. This is especially true for arteries which experience a dynamic pressure environment and stretch to provide arterial hydrostatic pressure.

Additionally, many ECMO runs span several weeks, leading to degradation of the vessel and surrounding tissue. Degradation of the vessel makes suturing the artery even more difficult and in some cases risky.

Another current option for post-ECMO procedures includes ligation of the artery to prevent bleeding from the vessel. However, ligation results in irreversible loss of the carotid artery, which can be problematic or impossible for some patients. For example, ligation is not possible for patients with a non-intact Circle of Willis. There is also the potential to require a second run of ECMO may be required, and ligation of the vessel requires that the patient have the vessel repaired instead of ligated on the second run or would have to undergo a sternotomy to place the cannulas directly int the heart and aorta. Ligation also is risky as patient's may develop age-related stenosis of the contralateral artery, severely limiting perfusion to the brain.

Another difficulty with vasculature outside of vascular access procedures is when the vasculature is damaged. Vascular damage is a leading cause of morbidity and mortality in patients with traumatic injuries. Damage to an artery can result in impaired blood flow to vital organs or limb ischemia. Approximately half of patients with an abdominal or pelvic vascular injury will develop multiorgan failure, and one quarter of them will die from their injuries. Arterial injuries to the limbs lead to amputation in 16.8% of patients and nerve damage in an additional 10.3%.

Current standard of care involves repairing the vascular defect with sutures. However, these repairs often require specialized expertise in vascular surgery. Additionally, several complications can arise, including stenosis, hemorrhage, thrombosis, or aneurysm. Stenosis typically occurs due to thickening of the smooth muscle cells in the blood vessel wall (neointimal hyperplasia) and inflammation as part of the healing process. Hemorrhage can occur due to incomplete sealing of the vessel juncture, dehiscence secondary to high arterial pressures, or leakage from suture holes. Thrombosis can occur if a vascular anastomosis becomes twisted, placed under tension or compression, or if there are alterations in flow leading to turbulence or stasis. Lastly, different elasticities of the grafted artery and host artery can lead to excessive mechanical strain being placed on the vascular anastomosis, which can result in an aneurysm.

### SUMMARY

A vascular repair device includes a hydrogel substrate, a biodegradable adhesive, and a biodegradable mold. The vascular repair device facilitates quick, effective, and simple closure of a vessel. The vascular repair device enables surgeons and other medical care providers, as appropriate, to close vessels without advanced training in vascular repair procedures. The vascular repair device facilitates closure of the vessel without having to have a secondary procedure for removal of sutures or other components. The vascular repair device is a sutureless repair system that facilitates biological repair of the vessel for long-term, permanent repair. The vascular repair device facilitates repair of a vessel that has an aperture through the vessel wall and is otherwise compromised (e.g., thinning of the vessel wall, frangible vessel wall, etc.) that prevents or limits the effectiveness of repair via current vascular closure methods. The vascular repair device facilitates reliable closure of vessels under dynamic pressure environments and under hydrostatic pressure. The vascular repair device is a biopolymer based solution for repairing biological lumens.

According to one example, a vascular repair device includes a hydrogel substrate configured to be positioned at least partially around a target vessel; a biodegradable adhesive configured to be positioned at least partially around the hydrogel substrate; and a biodegradable mold defining a trough sized to accommodate at least a portion of the target vessel and configured to contain at least a portion of the biodegradable adhesive.

In some examples, the hydrogel substrate includes at least one of polyethylene glycol thiol and polyethylene glycol maleimide.

According to certain embodiments, the hydrogel substrate is hydrophilic.

According to certain embodiments, the vascular repair device further includes a cellulose mesh configured to receive the hydrogel substrate.

In certain cases, the biodegradable adhesive is an acrylate adhesive.

The biodegradable adhesive may include at least one of epoxidized acrylate, trimethylopropane triacrylate, ethanol, and lithium phenyl-2,4,6-trimethylbenzoylphosphinate.

In embodiments, the biodegradable adhesive may be configured to biodegrade within about three months of being implanted in vivo.

The biodegradable adhesive may be curable in vivo.

The biodegradable adhesive may be photocurable.

The biodegradable mold may be an acrylate mold.

In certain embodiments, the biodegradable mold may include at least one of a cellulose mesh, polyethylene glycol diacrylate, and lithium phenyl-2,4,6-trimethylbenzoylphosphinate.

In certain examples, the biodegradable mold is hydrophobic.

The biodegradable mold may be configured to biodegrade within 6 months of implantation in vivo.

In an embodiment, a vascular repair kit is provided that includes a hydrogel substrate including at least one of polyethylene glycol thiol and polyethylene glycol maleimide; a biodegradable adhesive including at least one of epoxidized acrylate, trimethylopropane triacrylate, ethanol, and lithium phenyl-2,4,6-trimethylbenzoylphosphinate; and a biodegradable mold including at least one of a cellulose mesh, polyethylene glycol diacrylate, and lithium phenyl-2,4,6-trimethylbenzoylphosphinate.

In certain embodiments, the kit includes a cellulose mesh.

The kit may include a photoactivator.

A method of repairing a vessel is also disclosed that includes applying a hydrogel substrate to a vessel having an aperture defined through a vessel wall such that the hydrogel substrate is positioned covering the aperture on an exterior side of the vessel; applying a biodegradable adhesive to at least a portion of the hydrogel substrate proximate the aperture; and curing the biodegradable adhesive.

In certain embodiments, the method may include positioning a biodegradable mold about at least a portion of the vessel proximate the aperture, wherein the biodegradable mold is configured to support the biodegradable adhesive prior to curing.

In certain embodiments, the step of curing the biodegradable adhesive includes exposing the biodegradable adhesive to light having a predefined characteristic operable to instigate curing of the biodegradable adhesive.

In certain embodiments, the method may include wrapping the vessel with a cellulose mesh prior to applying the hydrogel substrate.

The foregoing embodiments are just examples and should not be read to limit or otherwise narrow the scope of any of the inventive concepts otherwise provided by the instant disclosure. While multiple embodiments are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is an illustration of an extracorporeal membrane oxygenation procedure, in accordance with an embodiment;

FIG. 1B is an illustration of various complications that can result from traditional suture-based vascular repair;

FIG. 2 is an illustration of a vascular repair device implanted about a vessel, in accordance with an embodiment;

FIG. 3 is a cross section of a vascular repair device implanted about a vessel, in accordance with an embodiment;

FIG. 4 is a cross section of another vascular repair device implanted about a vessel, in accordance with an embodiment;

FIG. 5 is a cross section of a vascular repair device including a mesh implanted about a vessel, in accordance with an embodiment;

FIG. 6 is a cross section of a vascular repair device including a hydrogel substrate that is positioned over the opening but not wrapped around a vessel, in accordance with an embodiment;

FIG. 7 is an illustration of a method of implanting a vascular repair about a vessel, in accordance with an embodiment;

FIG. 8 are photographs illustrating an example of a vascular repair device in use over a period of time, in accordance with an embodiment;

FIG. 9 is a graph of test results of a bench test of the vascular repair device showing the average pressure over time, in accordance with an embodiment;

FIG. 10 is a graph of test results of a bench test of the vascular repair device showing the average flow rate over time, in accordance with an embodiment; FIG. 11A is an illustration of a vascular repair device being implanted on a subject, in accordance with an embodiment;

FIG. 11B are photographs of a vascular repair device procedure being performed in an animal study, in accordance with an embodiment;

FIG. 12 illustrates an exemplary method of implementing a vascular repair device, in accordance with an embodiment;

FIGS. 13A-13D illustrates a benchtop test and results, in accordance with an embodiment;

FIGS. 14A-14C illustrate ultrasound results of a vascular repair device used on the vasculature of a test subject, in accordance with an embodiment;

FIG. 15A-C illustrate control and experimental arteries that underwent histological staining and analysis with charted results, in accordance with an embodiment; and

FIG. 16A-16B illustrates histology stains of both rat liver and brain tissue after implantation, respectively, in accordance with some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

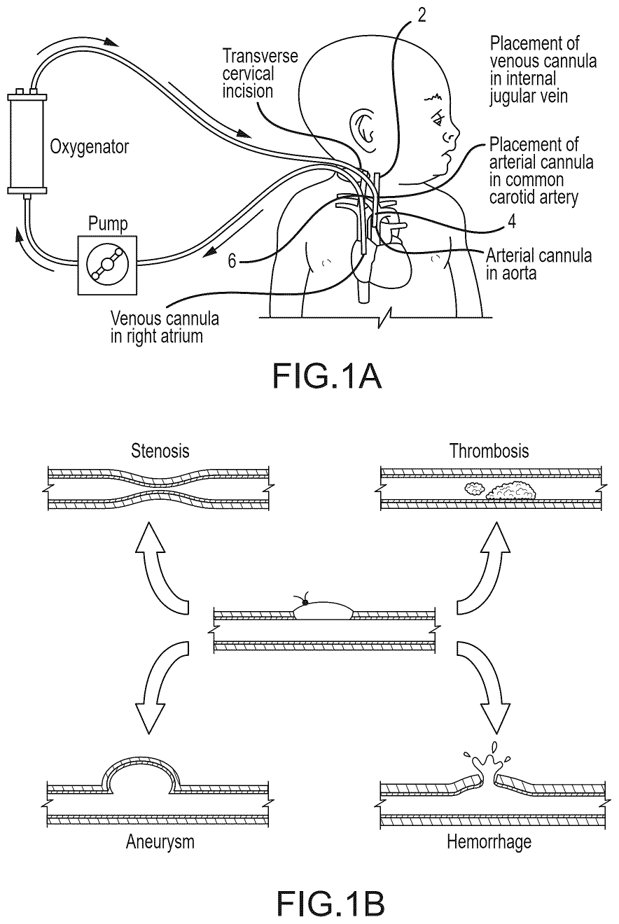

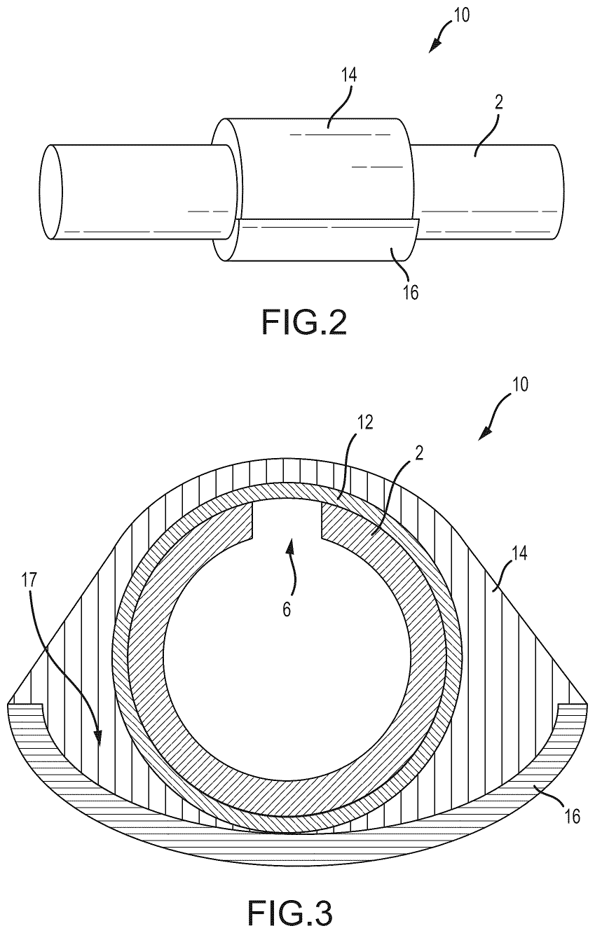

FIG. 1A illustrates an exemplary procedure in which a vessel 2 of a patient is cannulated. The vessel 2 in this example is cannulated in accordance with an extracorporeal membrane oxygenation (ECMO) procedure. In order to effectively oxygenate appropriate volumes of blood and to reduce stroke risk, large cannulas 4 are implemented in ECMO runs. After the vessel is decannulated, an opening 6 (e.g., a hole or aperture) (see FIG. 3) is left in the vessel, through which blood may escape. Closure of the vessel may be necessary after decannulation in order to limit hemorrhagic events. This is especially relevant for high-pressure dynamic vessels such as arteries, which are prone to hemorrhaging after cannulation. Additionally, traumatic arterial injuries are life threatening if not surgically repaired. However, traditional suture repair can be complex, skill-dependent, time-consuming, and result in complications such as bleeding, aneurysm, occlusion, and stenosis. FIG. 1B is an illustration of various complications that can result from traditional suture-based vascular repair. Provided herein are devices and methods for closure of vessels having a hole or aperture in the side wall of the vessels. More specifically, FIG. 2 illustrates a vascular repair device 10 for closing a vessel 2 having an opening 6 (see FIG. 3) through the vessel wall. The vascular repair device 10 facilitates quick, effective, and simple closure of the vessel. The vascular repair device 10 enables surgeons and other medical care providers, as appropriate, to close vessels without advanced training in vascular repair procedures. The vascular repair device 10 facilitates closure of the vessel without having to have a secondary procedure for removal of sutures or other components. The vascular repair device 10 is a sutureless repair system. The vascular repair device 10 facilitates biological repair of the vessel for long-term, permanent repair. The vascular repair device 10 facilitates repair of a vessel that has an aperture through the vessel wall and is otherwise compromised (e.g., thinning of the vessel wall, frangible vessel wall, etc.) that prevents or limits the effectiveness of repair via current vascular closure methods. The vascular repair device facilitates reliable closure of vessels under dynamic pressure environments and under hydrostatic pressure. The vascular repair device 10 is a biopolymer-based solution for repairing biological lumens. In some embodiments, the vascular repair device 10 is a bioabsorbable adhesive wrap that seals arterial defects (e.g., after traumatic injury), provides mechanical support during healing, and degrades into non-toxic byproducts. In some embodiments, the vascular repair device 10 may be applied in <5 minutes and consists of a hydrogel patch to cover the defect, a rapidly UV or other wavelength curable bioadhesive, and a generally U-shaped mold to localize the adhesive prior to curing.

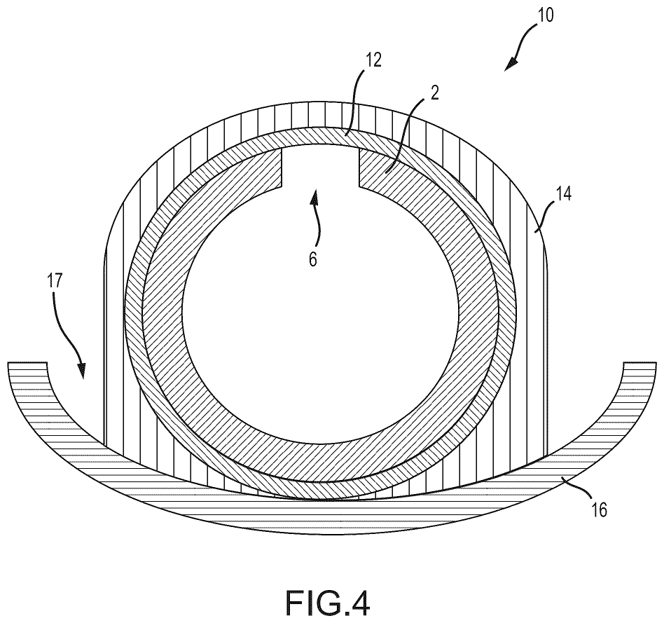

The vascular repair device 10 shown in FIG. 2 is provided as an example of the various features of the system and, although the combination of those illustrated features is clearly within the scope of invention, that example and its illustration is not meant to suggest the inventive concepts provided herein are limited from fewer features, additional features, or alternative features to one or more of those features shown in FIGS. 3 and 4.

Referring again to FIGS. 2-4, a vascular repair device 10 for repairing a vessel is illustrated. The vascular repair device 10 includes various components for facilitating closure of the aperture in the vessel. The vascular repair device 10 includes a hydrogel substrate 12, a biodegradable adhesive 14, and a biodegradable mold 16. The hydrogel substrate 12 configured to be positioned at least partially around the vessel 2 and at least over the opening 6 in the vessel, the biodegradable adhesive 14 is configured to be positioned at least partially around the hydrogel substrate, and the biodegradable mold 16 defines a trough sized to accommodate at least a portion of the vessel 2 and is configured to contain at least a portion of the biodegradable adhesive 14. These components operate together to facilitate closure and repair of the opening 6 of the vessel.

Turning first to a discussion of the hydrogel substrate 12, various properties and materials may be implemented as the hydrogel substrate 12. The hydrogel substrate 12 is configured to facilitate biological repair of the vessel 2 as well as limit intrusion of the biodegradable adhesive 14 into the vessel 2 via the opening 6. In some embodiments, the hydrogel substrate 12 may be hydrophilic. The hydrophilicity of the hydrogel substrate 12 may facilitate adhesion to the vessel and interact with the biodegradable adhesive 14, which in some embodiments may be hydrophobic, in order to restrict intrusion of the biodegradable adhesive 14 into the vessel 2 through the opening 6. The hydrogel substrate 12 is positioned over the opening 6 (e.g., on an exterior side) of the vessel 2 to cover the opening 6.

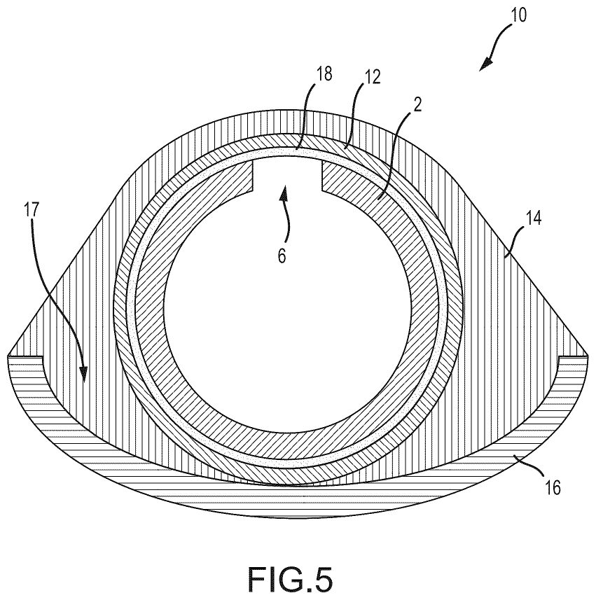

In some embodiments, the hydrogel substrate 12 includes least one of polyethylene glycol thiol and polyethylene glycol maleimide. It is understood that various other hydrogel substrates may be implemented in accordance within the spirit of this disclosure. In embodiments using polyethylene glycol thiol and polyethylene glycol maleimide, the two compounds may be mixed together which gel upon stirring. The polyethylene glycol thiol and polyethylene glycol maleimide mixture may then be coated (e.g., manually or otherwise) onto a mesh 18 (see FIG. 5), which is discussed hereafter. Polyethylene glycol thiol and polyethylene glycol maleimide may be implemented in some embodiments because of their biocompatible, biodegradable, and low toxicity. In some embodiments, additional compounds may be included or substituted in the hydrogel substrate to provide additional or alternative functionality. For example, various compounds with biological benefits may be added to reduce clotting, increase collagen synthesis, provide drug delivery, and so forth. In one example, the hydrogel substrate 12 may include calcium alginate in order to promote accelerate wound healing by improving collagen synthesis.

The biodegradable adhesive 14 is implemented in combination with the hydrogel substrate 12. The biodegradable adhesive 14 is operable to support the hydrogel substrate 12 to seal the opening 6 in the vessel 2 until the tissue in the vessel 2 can regenerate and close. The biodegradable adhesive 14 provides mechanical support to the hydrogel substrate 12 and provides a barrier through which fluids (e.g., blood and interstitial fluids) are unable to pass. The biodegradable adhesive 14 is operable to seal the hydrogel substrate 12 in place, to act as a hydrophobic barrier to fluid, and to provide mechanical support to prevent bleeding from the vessel 2. In some embodiments, the biodegradable adhesive 14 is an acrylate adhesive. However, it is understood that various compounds may be implemented within the spirit of this disclosure in order to prevent bleeding and to provide mechanical support of the vessel 2 during the healing process. In some embodiments, the biodegradable adhesive 14 is a mixture including epoxidized acrylate (EA) (including soybean epoxidized acrylate), trimethylol propane triacrylate (TMPTA) and ethanol. Lithium phenyl-2,4,6-trimethylbenzoylphosphinate (LAP) may also be dissolved within this mixture to allow for the mixture to harden upon UV light exposure. However, it is understood that additional curing agents may be added in order to cure and/or accelerate curing of the biodegradable adhesive, either by photoactivation, thermal activation, or otherwise.

In some embodiments implementing at least EA, TMPTA, and ethanol (and optionally LAP), the biodegradable adhesive 14 is able to adhere or stick readily upon contact with biological tissue (e.g., an exterior surface of the vessel 2) containing proteins. The biodegradable adhesive 14 may adhere to the vascular tissue in about 20 seconds. In those embodiments implementing LAP, the biodegradable adhesive 14 is exposed to ultraviolet light (or other suitable light to cause curing) for a about minute to ensure hardening of the biodegradable adhesive 14. Speaking more specifically about the composition of the biodegradable adhesive 14, EA may be the main component of biodegradable adhesive 14 and is viscous and hydrophobic, repelling water very easily. The viscosity of the biodegradable adhesive 14 allows for controlled placement of the biodegradable adhesive 14 on the vessel without collateral exposure of other tissue. The hydrophobicity of the biodegradable adhesive 14 also helps provide the fluid barrier for sealing the opening 6 of the vessel 2. TMPTA provides mechanical stiffness to the biodegradable adhesive 14 once UV-crosslinked. This provides stability to both the hydrogel substrate 12 and to the vessel during healing. This stability to the vessel 2 may be important especially in cases in which the vessel 2 is an artery and, accordingly, is subject to internal dynamic pressure. Additionally, the stability of the vessel 2 may be important in cases where there is additional damage or thinning of the vessel wall in order to allow healing of the vessel. Various ratios of EA and TMPTA may be varied for tunable mechanical properties. For example, a biodegradable adhesive 14 with a first ratio of EA to TMPTA result in a biodegradable adhesive 14 that is more hydrophobic but softer relative to a biodegradable adhesive 14 with relatively lower ratio of EA to TMPTA would result in a stiffer but more hydrophilic biodegradable adhesive 14. Additionally, a biodegradable adhesive 14 with a lower ration of EA to TMPTA may biodegradable more rapidly. Thus, the proportions of the various components may be altered in order to provide the appropriate conditions or parameters for various scenarios or implementations such as the healing time, vessel condition, hemodynamics and so forth may vary in different patients, across different ages, and in different anatomy. In some embodiments, the biodegradable adhesive 14 is provided such that it is configured to biodegrade within about three months of being implanted in vivo. This allows the vessel 2 to be supported for about three months as the vessel 2 heals.

In some embodiments, the biodegradable adhesive 14 is curable in vivo. This allows for the biodegradable adhesive 14 to be positioned by the health care provider as appropriate for the specific lesion and allows the biodegradable adhesive 14 to conform to the geometry of the vessel 2 prior to curing of the biodegradable adhesive 14. This allows the biodegradable adhesive 14 to provide a sufficient seal to the vessel 2 and the opening 6. As previously discussed, the biodegradable adhesive 14 may be cured in various ways depending on the specific properties of the parts used to form the compound. For example, those embodiments of the biodegradable adhesive 14 that implement LAP are photocurable. This allows the health care provider to control when and how the biodegradable adhesive 14 is cured on the vessel 2. The biodegradable adhesive 14 may be manipulated into an appropriate configuration for curing prior to initiation of the cure.

The vascular repair device 10 further includes the biodegradable mold 16. The biodegradable mold 16 is implemented in order to provide the biodegradable adhesive 14 in an appropriate configuration and/or to control the placement of the biodegradable adhesive 14 in vivo during application and prior to curing. Stated otherwise, the biodegradable mold 16 is used to localize the biodegradable adhesive only to the region of the vessel 2 where the opening 6 was made. This may be important as, in some embodiments, excessive amounts of biodegradable adhesive 14 may result in an inflammatory response and may occlude or alter anatomical positions of other vessels near to the vessel 2 being treated. This biodegradable mold 16 provides a surface around at least a portion of the vessel 2 which catches the biodegradable adhesive 14 during application and helps retain the adhesive about the vessel 2 until curing. The biodegradable mold 16 may define a trough 17 which is configured to receive a portion of the vessel 2 and to receive and retain a portion of the biodegradable adhesive 14. The biodegradable mold 16 may, for example, be positioned beneath a portion of the vessel 2 proximate or longitudinally aligned with the opening 6 such that a portion of the vessel 2 is positioned or received in the trough 17. The biodegradable adhesive 14 is then applied to the vessel 2 and received and controlled by the biodegradable mold 16 until curing of the biodegradable adhesive 14. The biodegradable mold 16 may be provided in various shapes and sizes depending on the anatomy of the patient, the viscosity of the biodegradable adhesive prior to curing, and so forth. While the mold is shown with side edges that extend only a portion of the way up the sides of the vessel, it is contemplated that the sides of the mold may configured to extend upward at least half the diameter of the vessel so as to better trap the biodegradable adhesive. Also, it should be readily apparent that the axial length of the mold should be at least, and more preferably longer than the axial size of the hole.

In some embodiments, the biodegradable mold 16 is an acrylate mold. The biodegradable mold 16 includes at least one of a cellulose mesh, polyethylene glycol diacrylate, and lithium phenyl-2,4,6-trimethylbenzoylphosphinate. In some embodiments, the biodegradable mold 16 includes a similar or the same formulation as the biodegradable adhesive 14. For example, the biodegradable mold 16 includes EA, TMPTA, and ethanol that is cured into the mold shape. The various formulations may be implemented for various purposes. For example, the EA, TMPTA, and ethanol formulation may be implemented when a relatively higher hydrophobicity and/or a slower biodegradation profile is needed. The biodegradable mold 16 is cured prior to insertion into the patient. The biodegradable mold 16 may be provided such that it is hydrophobic. The composition of the biodegradable mold 16 may be provided with various other compounds to provide the specific properties required by the health care provider. One some embodiments, the biodegradable mold 16 is configured to biodegrade within about six months (in some embodiments between six months and a year for full bidegradation) of implantation in vivo. In some embodiments, the mold may be configured so that it has some flexibility to permit it to be bent to better conform to the vessel after placement.

Various embodiments of vascular repair devices 10 are contemplated herein. Illustrations of such embodiments are provided and are not to be considered limiting to the shape and geometries that may be implemented. More specifically, FIGS. 3-5 illustrate various, non-limiting embodiments. FIG. 3 illustrates a vascular repair device 10 implementing a hydrogel substrate 12, a biodegradable adhesive 14, and a biodegradable mold 16. The biodegradable adhesive 14 is provided so as to fill the remainder of the trough 17 that is not occupied by the vessel. Small amounts of biodegradable adhesive may be positioned or seep between the biodegradable mold 16 and the hydrogel substrate 12. FIG. 4 illustrates an embodiment in which the trough 17 is not entirely filled. FIG. 5 illustrates an embodiment in which a mesh 18 is implemented. It is understood that various combinations of these features may be implemented in combination with each other and other features as discussed herein.

In some embodiments, the vascular repair device 10 is provided in a vascular repair kit. The kit includes the a hydrogel substrate 12 (e.g., including at least one of polyethylene glycol thiol and polyethylene glycol maleimide), a biodegradable adhesive 14 (e.g., including at least one of epoxidized acrylate, trimethylopropane triacrylate, ethanol, and lithium phenyl-2,4,6-trimethylbenzoylphosphinate), and a biodegradable mold 16 (e.g., including at least one of a cellulose mesh, polyethylene glycol diacrylate, and lithium phenyl-2,4,6-trimethylbenzoylphosphinate). The kit can include any of the features as previously discussed with respect to the vascular repair device, including, but not limited to a mesh 18 (e.g., cellulose mesh) and a photoactivator (e.g., a light source, not shown). The kit may also include applicators, clamps, and other implements for effectuating vascular closure. The kit may be sterilized and packaged. The various component parts may be provided separate, including the compounds used for the hydrogel substrate 12 and the biodegradable adhesive 14, such that the medical professional can mix the component parts for the appropriate levels of viscosity, biodegradation timelines, and so forth. However, some kits may be provided ready for use off the shelf.

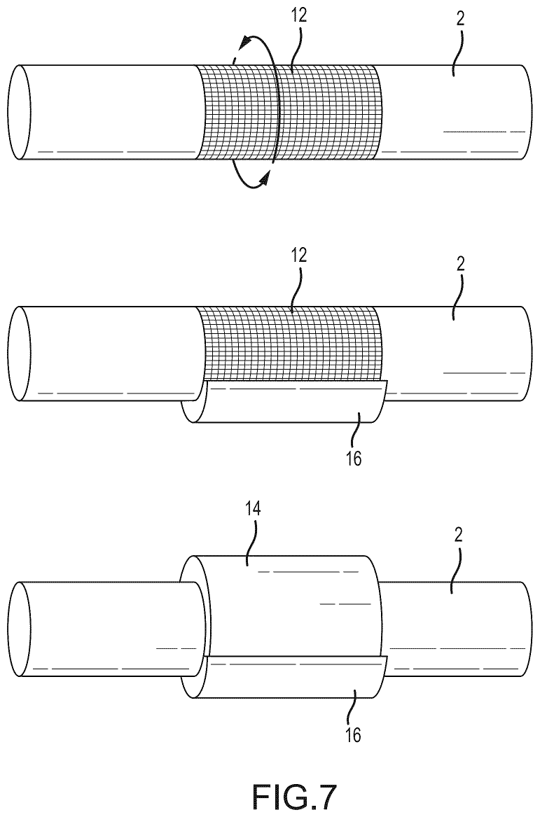

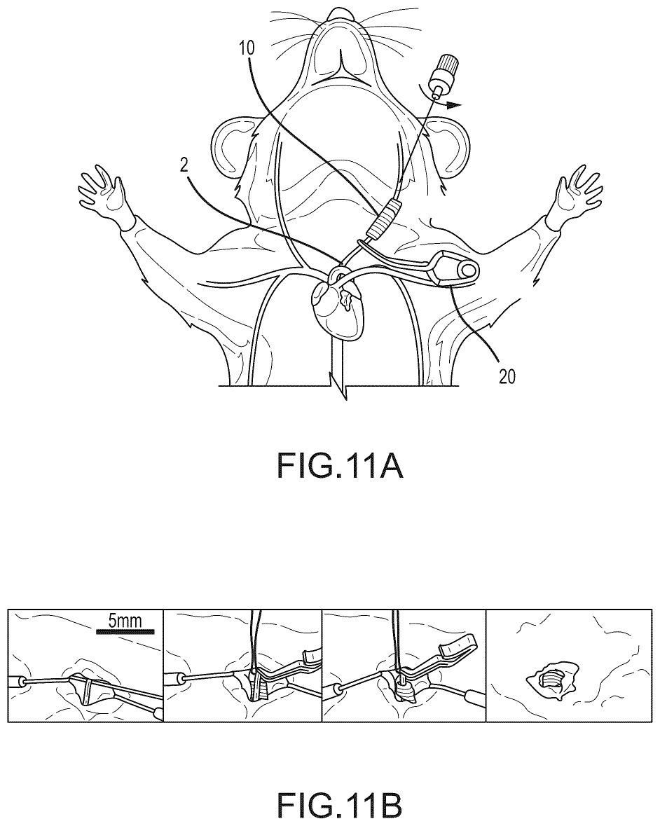

A method of repairing a vessel 2 (see FIG. 7) includes applying a hydrogel substrate 12 to a vessel 2 having an aperture 6 defined through the vessel wall such that the hydrogel substrate 12 is positioned covering the aperture 6 on an exterior side of the vessel 2, applying a biodegradable adhesive 14 to at least a portion of the hydrogel substrate 12 proximate the aperture 6, and curing the biodegradable adhesive 14. The method may include positioning a biodegradable mold 16 about at least a portion of the vessel 2 proximate the aperture 6, wherein the biodegradable mold 16 is configured to support the biodegradable adhesive 14 prior to curing. When curing the biodegradable adhesive 14, the biodegradable adhesive is exposed to light having a predefined characteristic that is operable to instigate curing of the biodegradable adhesive. The predefined characteristics may include wavelength, luminosity, and so forth. In some embodiments, the method includes providing a cellulose mesh 18 to which the hydrogel substrate 12 is applied. The cellulose mesh 18 is wrapped about the vessel 2 either prior to positioning the hydrogel substrate 12 with the vessel 2 or the hydrogel substrate 12 is applied to the cellulose mesh 18 and then the hydrogel substrate 12 and cellulose mesh 18 are positioned on the vessel. In order to apply the vascular repair device 10 to the vessel 2, the method may include clamping the vessel 2 to temporarily prevent blood flow through the vessel 2 and releasing the clamp 20 (see FIG. 11A) after the curing is completed. In some embodiments, a complete procedure and the order of the procedure may include clamping the vessel 2 to temporarily prevent blood flow through the vessel, placing the biodegradable mold 16 beneath the vessel 2, placing the biodegradable mold 16 beneath the vessel 2, positioning the hydrogel substrate 12 (optionally including the cellulose mesh 18) on top of or around the vessel 2 where the opening 6 is located, adding biodegradable adhesive 14 to the top of the vessel 2 (and optionally letting the biodegradable adhesive 14 set for 20 seconds to allow for the adhesive to adhere to the vessel 2 and hydrogel substrate 12, exposing the biodegradable adhesive 14 to ultraviolet light to allow for crosslinking to take place (around one minute of exposure), and unclamping the vessel 2 to allow normal blood flow through the artery. The vascular repair device 10 will be bioabsorbable over several weeks as the arterial defect/lesion heals.

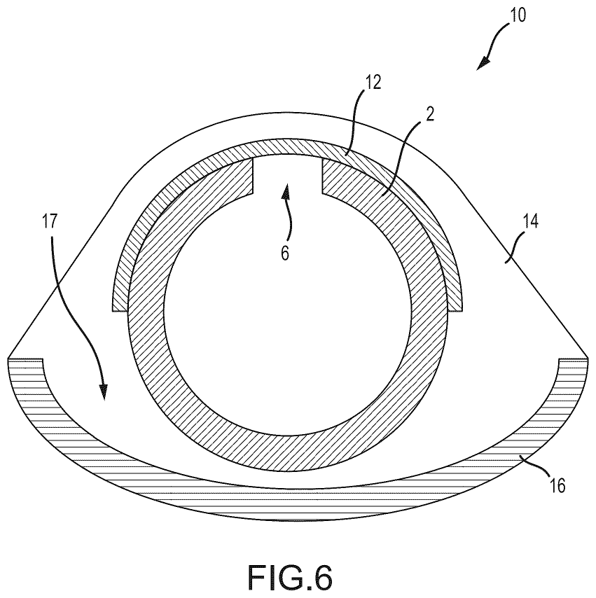

In some embodiments, access around the entire vessel 2 is not possible or is difficult. In those embodiments, the hydrogel substrate 12 is positioned across the opening 6 and not wrapped around the entire circumference of the vessel (see FIG. 6). The biodegradable adhesive 14 can then be applied as previously discussed herein.

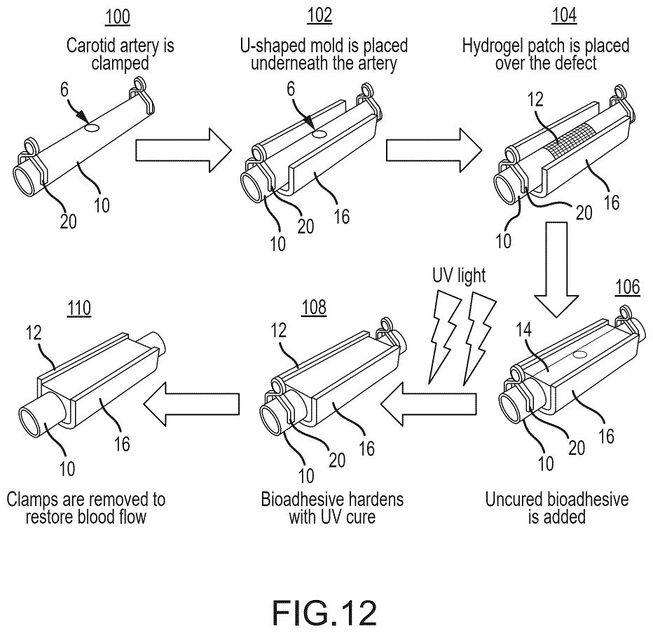

FIG. 12 provides an exemplary procedure for treating a vessel 2 with an opening 6 therethrough. Although a carotid artery is shown here, it is understood that the procedure and vascular repair device 10 may be implemented with a variety of vessels. Step 100 include clamping the vessel 2. The clamps 20 are used to temporarily cut off blood flow to the region of the vessel with the arterial defect. Step 102 includes positioning a biodegradable mold 16 around a portion of the vessel 2. The biodegradable mold 16 serves to limit or prevent excessive biodegradable adhesive 14 from leaking to surrounding tissue. Step 104 includes positioning a hydrogel substrate 12 across or over the opening 6. This helps to cover the arterial defect and aid in healing. Step 106 includes positioning a biodegradable adhesive 14 that is uncured with the biodegradable mold 16 and the hydrogel substrate 12. The biodegradable adhesive 14 is positioned to encase the vessel 10 and hydrogel substrate 12. Step 108 includes curing the biodegradable adhesive 14; for example, hardening the biodegradable adhesive 14 with UV light. The hardened biodegradable adhesive 14 is mechanically strong, enabling it to withstand supraphysiologic blood pressures. Additionally, in some embodiments the biodegradable adhesive 14 is hydrophobic, which repels blood from the arterial defect and prevents hemorrhaging. Step 110 includes removing the clamps 20 to restore blood flow. The entire procedure may take approximately 5 minutes.

Compared to various alternatives, the vascular repair device 10 may have numerous benefits. Unlike suturing, it can be quickly implemented (<10 mins) and does not require a specialized vascular surgeon. Despite its ease of use, the wrap was mechanically robust, demonstrating an average burst pressure more than 10 times a typical mean arterial blood pressure an important benefit over competing patches, adhesives, or plugs. Additionally, unlike stents or intraluminal devices, it is applied around the outside of the vessel, allowing it to be used in small arteries such as the 1 mm diameter as seen in the examples below including rat carotid arteries. The vascular repair device 10 components are fully customizable and can be adapted to large or small arteries. Finally, the components of the bioadhesive wrap are biocompatible and biodegradable. In the long-term rat study included below, the materials did not cause detectable inflammation or cytotoxicity, and there was evidence of biodegradation at 4 weeks.

### Test Methods

It should be understood that although certain methods and equipment are described below, other methods or equipment determined suitable by one of ordinary skill in the art may be alternatively utilized.

### Testing

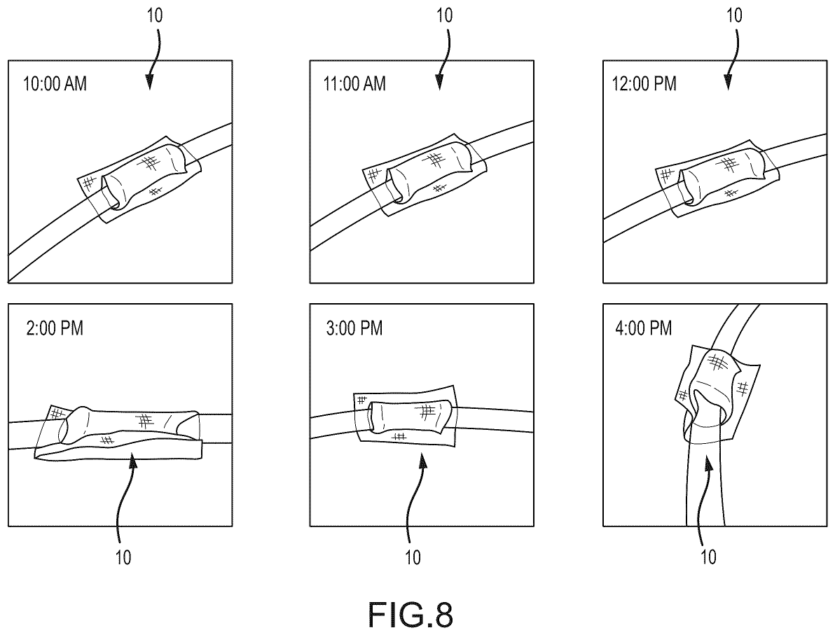

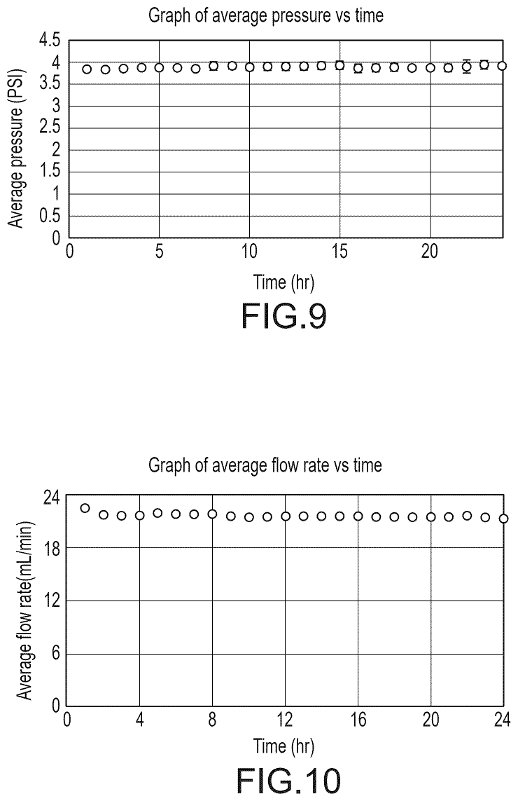

A test was performed to determine the efficacy of sealing a hole through a side wall of a tubular structure. A peristaltic pump including an inlet tube and an outlet tube was implemented. The inlet and outlet tubes received and delivered fluid from and to a fluid reservoir. A pressure gauge was coupled to one of the tubes and a hole was formed through the side wall of one of the tubes. The vascular repair device 10 was used to repair the hole in the side wall of the tube. The peristaltic pump was run for a 24-hour period. FIG. 8 shows the vascular repair device 10 at different periods. Minimal changes were observed in the overall structure of the vascular repair device 10 over the testing period. No leakage of any fluid was detected during the 24-hour period. The pressure over the 24-hour period was recorded and showed a constant pressure over the testing period, indicative of no leaks (See FIG. 9). Flow rate over time was also recorded and showed a substantially constant flow rate over the 24-hour period (see FIG. 10).

In another test, the vascular repair device 10 was tested on rats over a two-hour period. In a sample of five trials, the vascular repair device 10 was implanted on each subject as previously provided herein. All five subjects survived two hours with the vascular repair device 10 implanted and never showed any signs of hemorrhaging. The average oxygen saturation and heart rate of the subjects were stable and within the normal physiological parameters for the subjects (80%-100% oxygen saturation and 200-300 beats per minute. A table of the average values over the two-hour testing period is provided below.

TABLE 1

| | Average Oxygen | Average Heart Rate |
|---|---|---|
| Time (min) | Saturation(%) | (bpm) |
| 0 | 88.6 | 240.4 |
| 30 | 87.8 | 234.2 |
| 60 | 89.6 | 220 |
| 90 | 86.8 | 219.8 |
| 120 | 83.2 | 227 |

Peristaltic Pump Tests with Tubing and Porcine Carotid Arteries

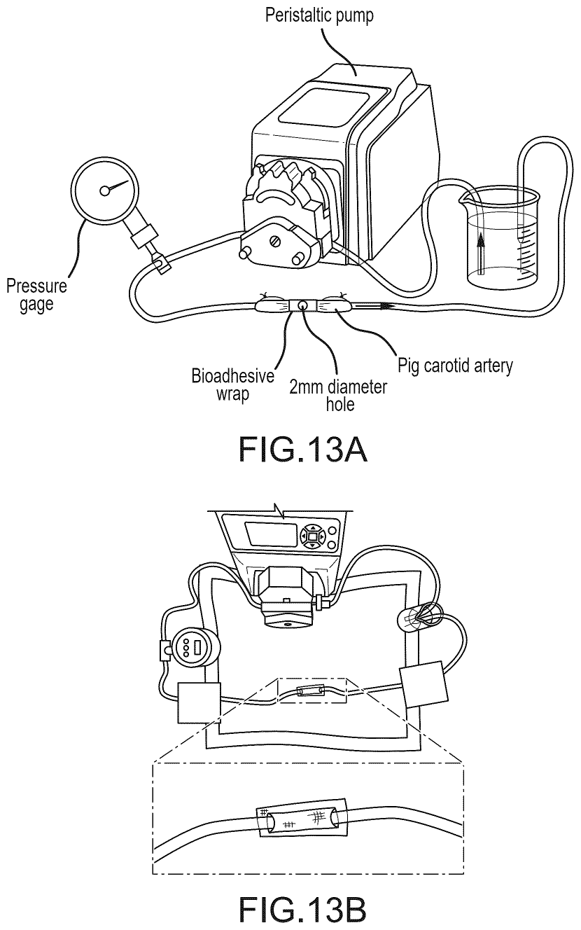

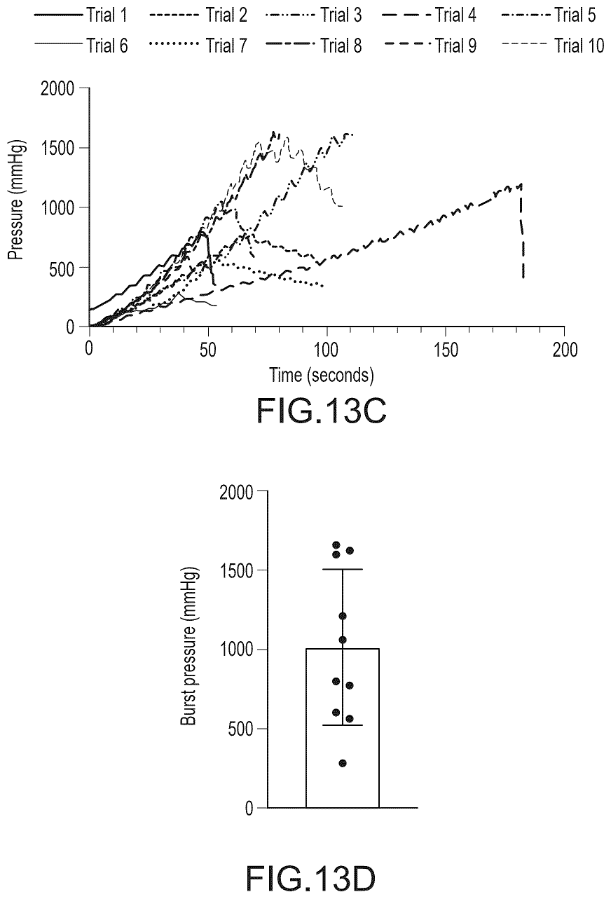

In a benchtop test, the mechanical properties of the bioadhesive wrap using a benchtop flow circuit (see FIGS. 13A-D). FIG. 13A is a schematic of benchtop flow circuit. FIG. 13B is a photograph of benchtop flow circuit with bioadhesive wrap implemented on a porcine carotid artery. FIG. 13C illustrates graphs of pressure over time for burst pressure trials. The burst pressure was taken to be the maximum pressure achieved prior to either wrap leaking or loss of pressure. FIG. 13D is a summary of burst pressure results. Points denote individual trial results. The bar graph denotes mean±standard deviation.

The mechanical integrity of the wrap was tested using a benchtop mock circuit loop. For the continuous flow tubing test, a 2 mm diameter hole was cut into ⅛″ inner diameter (ID) tubing. The wrap was then applied over the defect; the adhesive component was added in two layers with 800 μL of bioadhesive used for each layer. Water (dyed blue for easy visualization) was pumped through the circuit using a Peristaltic Pump. The flow rate was increased to a target pressure of 200 mmHg, and the circuit was monitored for leaking for 24 hours. The porcine carotid artery test used a similar setup. The carotid artery from an adult pig was connected to ⅛″ ID tubing via 16-gauge catheters inserted into either end of the artery and held in place with sutures wrapped around the outside of the artery. A 2 mm hole was cut into the artery, and dyed water was pumped at a target pressure of 200 mmHg. The circuit was monitored for 4 hours for leaking. For the max burst pressure test, the defect and tubing used was identical to the continuous flow tubing test, but the tubing was primed and then clamped on one end. The dyed water was then pumped at a flow rate of 2 ml/min to steadily increase the pressure until the mechanical failure of the wrap. The burst pressure was defined as the maximum pressure withstood without leaking (see FIG. 13D). The mean±standard deviation (SD) burst pressure was 1017±493 mmHg (n=10). The minimum burst pressure was 279 mmHg, well above physiological arterial pressures. The vascular repair device 10 the withstood pressures 10 times that of typical arterial pressures (Burst pressure: 1017±493 mmHg, mean±standard deviation, n=10).

### Animal Studies

FIG. 11B shows the vascular repair device 10 being used in vivo in an animal study. In animal testing, the vascular repair device 10 was included in two different studies.

### Short Term Rat Carotid Artery Experiments (2 Hours)

In the first study, after induction of anesthesia, exposing the right carotid artery (˜0.7-1 mm diameter) by a surgical cutdown procedure. A ˜0.7 mm defect was made in the rat carotid artery. The vascular repair device 10 was applied, and the rats were observed for 2 hours. The wrap was simple to implement, and the entire procedure took approximately 5 minutes. None of the rats died or had any bleeding complications. However, 2 of the 10 rats had a vascular occlusion as determined by no blood flow distal to the wrap, thought to be related to too much uncured adhesive being added at a single time. Based on this result, future implementations used a decreased amount of adhesive (from 10 μL down to 5 μL). Additionally, the adhesive was added and cured in multiple layers.

### Long Term Rat Carotid Artery Experiments (4 Weeks)

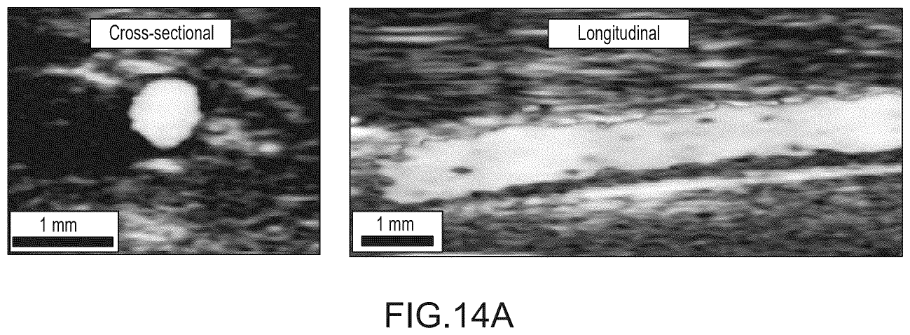

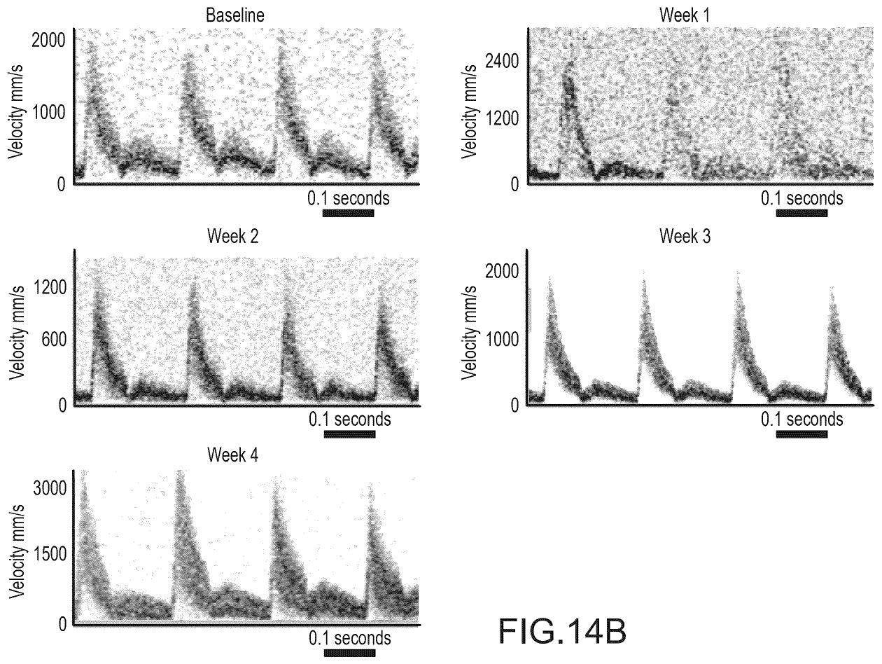

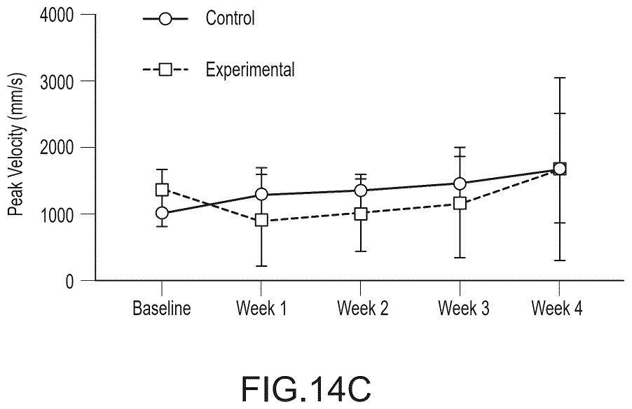

Following the short-term in vivo study, a long-term survival study was conducted to assess the wrap's efficacy over time. Adult rats (400-500 g, n=8) underwent anesthesia and surgical cutdown to expose the right carotid artery. A slightly larger defect (˜0.9 mm) was made, and the wrap was applied. The skin incision was closed with suture, and the rat was allowed to recover. The study continued for up to 4 weeks. One rat developed a hematoma on postoperative day 1 and was euthanized. The remaining 7 had no bleeding complications. Vascular patency was evaluated over time using Doppler ultrasound (see FIG. 14a). None of the rats had complete occlusion of the vessel, though there was subjective evidence of partial occlusion or stenosis in 2 of the 8 rats. In addition, a pulsed wave Doppler ultrasound was used to quantify the flow velocity through the artery over time and compare it to the contralateral carotid artery, which served as an internal control (see FIG. 14B). A normal pulsed wave Doppler graph follows a triphasic blood flow pattern starting with a sharp increase in blood velocity representing systole, followed by a decrease in blood flow velocity to below baseline. The vascular repair device 10 preserved a normal triphasic blood flow velocity profile and had no effect on peak systolic velocity when compared to an uninjured control artery.

Referring to FIGS. 14B-14C, the results of the Doppler ultrasound are provided. Representative pulsed wave Doppler ultrasound graphs for an experimental carotid artery at baseline and weeks 1-4 following implantation of bioadhesive wrap. Additionally, a quantified peak blood flow velocity of both control and experimental rat carotid arteries is provided. A baseline peak velocity was 1018±202 and 1361±303, respectively. At week one, the peak velocity was 1287±404 and 903±701, respectively. At week two, the peak velocity was 1364±164 and 1021±579, respectively. At week three, the peak velocity was 1475±385 and 1166±840, respectively. At week four, the peak velocity was 1697±811 and 1673±1370 mm/s, respectively. A mixed effects analysis with fixed effects of time, treatment, and time x treatment as an interaction term was performed. There was no significant difference in peak systolic velocity due to treatment (p=0.58), time (p=0.24), or time x treatment (=0.38). Post-hoc analysis using Tukey's test for multiple comparisons saw no differences between the experimental and control arteries at any time point (p=0.38, 0.33, 0.38, 0.49, and 0.69 at baseline, week 1, week 2, week 3, and week 4).

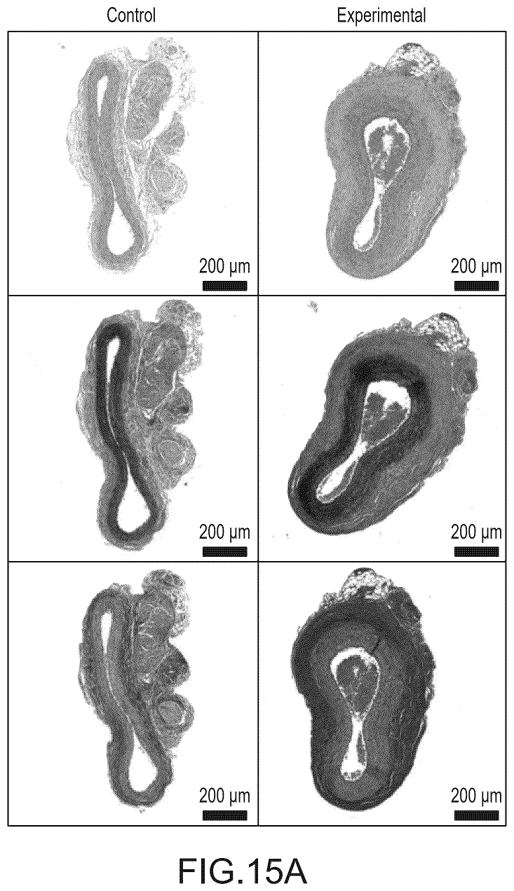

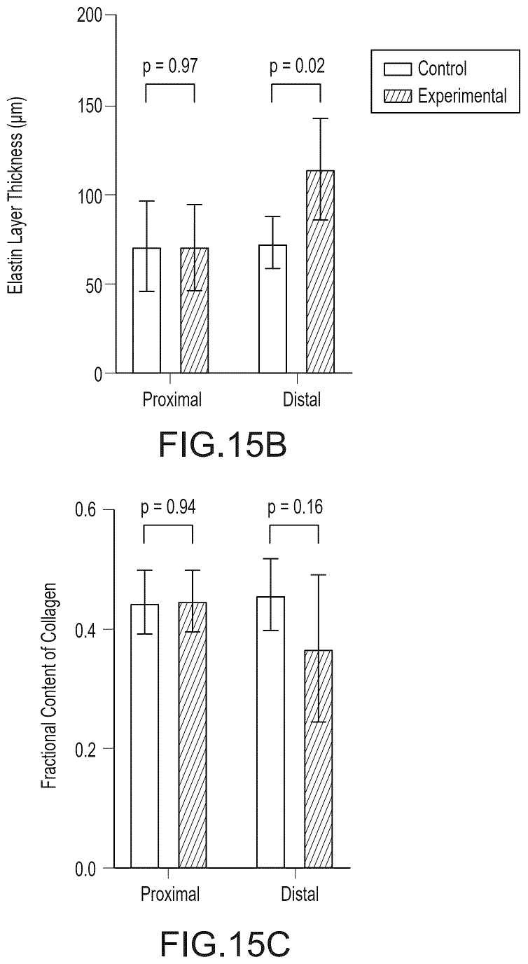

At the end of the study, the rats were euthanized, and both control and experimental arteries underwent histological staining and analysis (see FIG. 15). Each artery was divided into its proximal and distal half, which were analyzed separately. FIG. 15 shows the histology results of hematoxylin and eosin (H&E; general cellular structure stain), Verhoeff (elastin stain), and Masson's trichrome (collagen stain) stains for both the control and experimental arteries. Regarding FIG. 15, the top images are an H&E, the middle images are Verhoeff, and the bottom images are Masson Trichrome stains of both control (left) and experimental (right) rat carotid arteries. The graphs illustrated in FIGS. 15B-15C demonstrate elastin layer thickness and fractional collagen content of both control and experimental arteries for both proximal and distal halves of the artery. H&E revealed an absence in inflammatory cells (e.g neutrophils) for all experimental and control arteries. The elastin layer thickness was 70.8±24.2 μm (mean±SD) in the proximal experimental arteries versus 71.2±25.2 μm in the proximal control arteries (p=0.97). By contrast, the elastin layer was thickened in the distal experimental arteries (114.4±28.5 μm versus control 73.6±14.8, p=0.02). The fractional collagen content for the proximal arteries was 0.445±0.054 and 0.448±0.052 for the control and experimental arteries, respectively (p=0.94). In the distal arteries, the collagen content was 0.459±0.060 and 0.368±0.124, respectively (p=0.16).

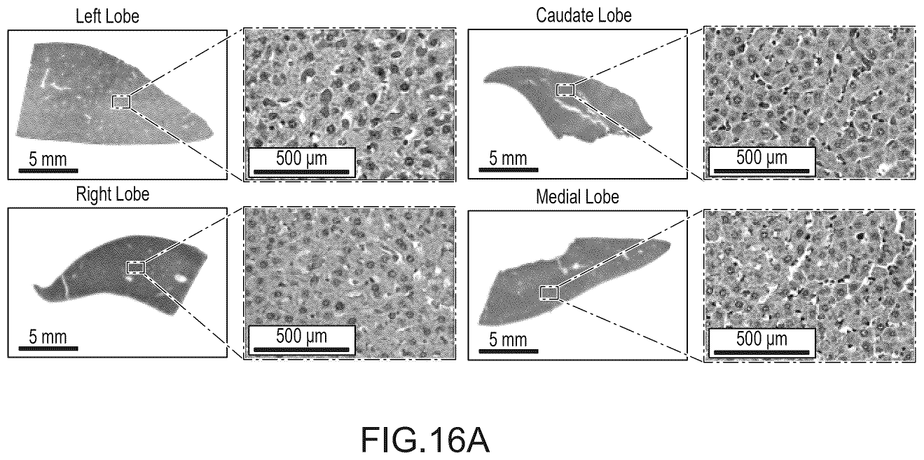

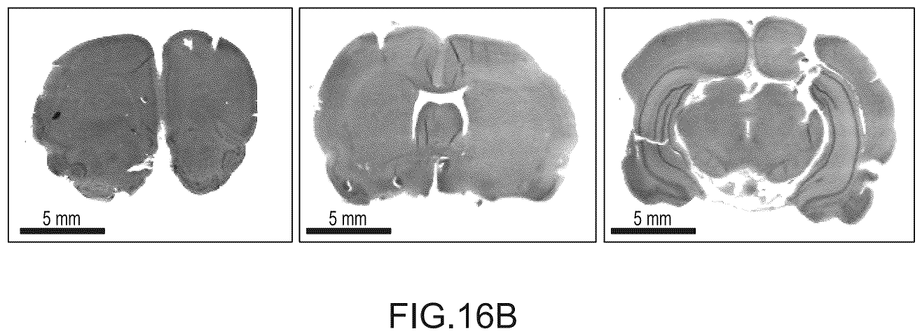

In addition to histological analysis, the liver and brain were analyzed for evidence of toxicity (see FIG. 16A-16B). The liver is the site of clearance for the wrap's breakdown products. The liver was stained with H&E and found no evidence of inflammation or necrosis. FIGS. 16A-B includes histology stains of both rat liver (FIG. 16A) and brain tissue (FIG. 16B). Referring to the specific images, “a” represents H&E stains of left, caudate, right and medial lobes of the liver and “b” represented cresyl violet stains of coronal brain slices.

### Materials

The materials provided here are not exclusive of other materials that may be implemented in conjunction with the vascular repair device 10 and are not intended to be limiting to the discussion and disclosures provided herein. The hydrogel substrate 12 consisted of an oxidized regenerated cellulose mesh coated with a 4 mM mixture of polyethylene glycol (PEG)-maleimide and PEG-thiol. The size of the hydrogel substrate 12 was adjusted such that the edge length of the square patch approximately matched the circumference of the target vessel. To create the bioadhesive, a mixture of 46% (w/w) soybean oil epoxidized acrylate (SEA; Sigma Aldrich), 27% trimethylolpropane triacrylate (TMPTA; Sigma Aldrich), 9% lithium phenyl-2,4,6-trimethylbenzoylphosphinate (LAP, Sigma Aldrich), and 18% ethanol was combined into solution. The biodegradable adhesive 14 was cured by applying 365 nm UV light at an intensity of 1.2 W/cm² for 30 seconds. 2 layers of biodegradable adhesive 14 were added for each surgical procedure to improve mechanical stability of the wrap. The U-shaped mold was shaped using a 3D printed trough. The radius and length of the trough (and thus the mold) depended on the size of the target vessel. For the tubing and benchtop artery testing, a larger trough was used (5 mm radius, 4.5 cm length). For the rat studies with a smaller artery, a smaller trough was used (1.5 mm radius, 1 cm length). Cellulose mesh was lined along the template, and biodegradable adhesive 14 solution was added in a thin layer. A rectangular piece of a cellulose acetate filter was cut to size and pressed into the mold, and UV light was applied to cure the biodegradable adhesive 14—approximately 1 minute.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A vascular repair device comprising:
a hydrogel substrate configured to be positioned at least partially around a target vessel;
a biodegradable adhesive configured to be positioned at least partially around the hydrogel substrate; and
a biodegradable mold defining a trough sized to accommodate at least a portion of the target vessel and configured to contain at least a portion of the biodegradable adhesive.

2. The vascular repair device of claim 1, wherein the hydrogel substrate includes at least one of polyethylene glycol thiol and polyethylene glycol maleimide.

3. The vascular repair device of claim 1, wherein the hydrogel substrate is hydrophilic.

4. The vascular repair device of claim 1, further comprising a cellulose mesh configured to receive the hydrogel substrate.

5. The vascular repair device of claim 1, wherein the biodegradable adhesive is an acrylate adhesive.

6. The vascular repair device of claim 1, wherein the biodegradable adhesive includes at least one of epoxidized acrylate, trimethylopropane triacrylate, ethanol, and lithium phenyl-2,4,6-trimethylbenzoylphosphinate.

7. The vascular repair device of claim 1, wherein the biodegradable adhesive is configured to biodegrade within about three months of being implanted in vivo.

8. The vascular repair device of claim 1, wherein the biodegradable adhesive is curable in vivo.

9. The vascular repair device of claim 8, wherein the biodegradable adhesive is photocurable.

10. The vascular repair device of claim 1, wherein the biodegradable mold is an acrylate mold.

11. The vascular repair device of claim 1, wherein the biodegradable mold includes at least one of a cellulose mesh, polyethylene glycol diacrylate, and lithium phenyl-2,4,6-trimethylbenzoylphosphinate.

12. The vascular repair device of claim 1, wherein the biodegradable mold is hydrophobic.

13. The vascular repair device of claim 1, wherein the biodegradable mold is configured to biodegrade within six months of implantation in vivo.

14. A vascular repair kit comprising:
a hydrogel substrate including at least one of polyethylene glycol thiol and polyethylene glycol maleimide;
a biodegradable adhesive including at least one of epoxidized acrylate, trimethylopropane triacrylate, ethanol, and lithium phenyl-2,4,6-trimethylbenzoylphosphinate; and
a biodegradable mold including at least one of a cellulose mesh, polyethylene glycol diacrylate, and lithium phenyl-2,4,6-trimethylbenzoylphosphinate.

15. The vascular repair kit of claim 14, further comprising a cellulose mesh.

16. The vascular repair kit of claim 14, further comprising a photoactivator.

17. A method of repairing a vessel, comprising:
applying a hydrogel substrate to a vessel having an aperture defined through a vessel wall such that the hydrogel substrate is positioned covering the aperture on an exterior side of the vessel;
applying a biodegradable adhesive to at least a portion of the hydrogel substrate proximate the aperture; and
curing the biodegradable adhesive.

18. The method of claim 17, further comprising positioning a biodegradable mold about at least a portion of the vessel proximate the aperture, wherein the biodegradable mold is configured to support the biodegradable adhesive prior to curing.

19. The method of claim 17, wherein curing the biodegradable adhesive includes exposing the biodegradable adhesive to light having a predefined characteristic operable to instigate curing of the biodegradable adhesive.

20. The method of claim 17, further comprising wrapping the vessel with a cellulose mesh prior to applying the hydrogel substrate.
